(19) United States
(12) Patent Application Publication
(10) Pub. No.: US 2026/0251745 A1
(43) Pub. Date: Aug. 27, 2026

(54) UNDER-SAMPLING K-SPACE FOR A THREE-DIMENSIONAL ECHO PLANAR IMAGING (EPI) SCAN WITH MAGNETIC RESONANCE

(71) Applicant: GE Precision Healthcare LLC, Waukesha, WI (US)

(72) Inventors: Rafael Brada, Tel Aviv-Yafo (IL); Sangtae Ahn, Guilderland, NY (US); Tim Sprenger, Munich (DE); Brice Fernandez, Montrouge (FR)

(73) Assignee: GE Precision Healthcare LLC, Waukesha, WI (US)

(21) Appl. No.: 19/063,440

(22) Filed: Feb. 26, 2025

(51) Int. Cl. G01R 33/561

(52) U.S. Cl. CPC G01R 33/5616

(57) ABSTRACT

A control system and method for a magnetic resonance imaging (MRI) system controller is provided to sub-sample k-space for an MRI scan. The method includes, for each radio-frequency excitation pulse in the MRI scan, sub-sampling ky-kz planes using a three-dimensional (3D) echo planar imaging (EPI) sequence by coherently sub-sampling phase encodings in the ky dimension and incoherently sub-sampling phase encodings in the kz dimension. The coherent sub-sampling corresponds to a number of radio-frequency excitation pulses. The ky-kz planes represent a sub-sampling pattern in the ky-kz space, using an incoherent sub-sampling in the kz dimension.

### BACKGROUND

The present disclosure generally relates to systems and methods for magnetic resonance imaging. More particularly, the disclosure relates to systems and methods for under-sampling k-space for a three-dimensional (3D) EPI scan performed with magnetic resonance (MR).

MR imaging (MRI) has proven useful in diagnosis of many diseases. For example, MRI provides detailed images of soft tissues, abnormal tissues such as tumors, and other structures, which can be challenging for other imaging modalities, such as computed tomography (CT). Further, MRI operates without exposing patients to ionizing radiation experienced in modalities such as the x-rays used in CT scans. As such, MRI may be used to obtain internal physiological information about a patient through brain imaging, thoracic imaging, spine imaging, cardiac imaging, and imaging other sections or tissues within a patient's body.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect of the disclosure, a method includes, for each radio-frequency excitation pulse in a magnetic resonance imaging (MRI) scan, sub-sampling ky-kz planes using a three-dimensional (3D) echo planar imaging (EPI) sequence by coherently sub-sampling phase encodings in the ky dimension and incoherently sub-sampling phase encodings in the kz dimension. The coherent sub-sampling corresponds to a number of radio-frequency excitation pulses. The ky-kz planes represent a sub-sampling pattern in the ky-kz space, using an incoherent sub-sampling in the kz dimension.

In one embodiment, the incoherent sub-sampling in the kz dimension corresponds to a random sub-sampling pattern.

In another embodiment, the phase encodings in the kz dimension are the same phase encoding values in the kz dimension for each phase encoding in the ky dimension for a single radio-frequency excitation pulse.

In another embodiment, the phase encodings in the kz dimension are the same phase encoding values in the kz dimension for each phase encoding in the ky dimension for all radio-frequency excitation pulses.

In another embodiment, the incoherent sub-sampling is based on a one-dimensional variable density Poisson disk in the kz dimension.

In another embodiment, the ky-kz planes represent a sub-sampling pattern having a density in the ky dimension that varies as a function of kz.

In another embodiment, the incoherent sub-sampling is based on a two-dimensional variable density Poisson disk in the ky dimension and the kz dimension in at least a portion of a ky-kz space.

In one aspect of the disclosure, an MRI system includes a magnet system configured to generate a polarizing magnetic field about at least a portion of a subject arranged in the MRI system, gradient coils configured to apply gradient pulses to the polarizing magnetic field, a radio frequency (RF) system configured to apply an RF field to the subject and to acquire magnetic resonance (MR) image data therefrom, a processing device, and a memory storage device having instructions executable by the processing device to, for each radio-frequency excitation pulse of a plurality of radio-frequency excitation pulses in the MRI scan, sub-sample a plurality of ky-kz planes using a three-dimensional (3D) echo planar imaging (EPI) sequence by sub-sampling phase encodings using a coherent sub-sampling in a ky dimension and sub-sampling, for each of the phase encodings in the ky dimension, one or more phase encodings in a kz dimension. The coherent sub-sampling corresponds to a number of radio-frequency excitation pulses. Additionally, the ky-kz planes represent a sub-sampling pattern in the ky-kz space, using an incoherent sub-sampling in the kz dimension.

In one embodiment, the incoherent sub-sampling in the kz dimension corresponds to a pseudo-random sub-sampling pattern.

In another embodiment, the phase encodings in the kz dimension are the same phase encoding values in the kz dimension for each phase encoding in the ky dimension for a single radio-frequency excitation pulse.

In another embodiment, the phase encodings in the kz dimension are the same phase encoding values in the kz dimension for each phase encoding in the ky dimension for all the radio-frequency excitation pulses.

In another embodiment, the incoherent sub-sampling is based on a one-dimensional variable density Poisson disk in the kz dimension.

In another embodiment, the ky-kz planes represent a sub-sampling pattern having a density in the ky dimension that varies as a function of kz.

In another embodiment, the incoherent sub-sampling is based on a two-dimensional variable density Poisson disk in the ky dimension and the kz dimension in at least a portion of a ky-kz space.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 is a schematic diagram of an MRI system for under-sampling k-space for a three-dimensional (3D) EPI scan performed with magnetic resonance, according to some embodiments of the present disclosure.

FIG. 2A is a pulse sequence diagram representing one shot of a multi-shot EPI, according to some embodiments of the present disclosure.

FIG. 2B is a pulse sequence diagram representing one shot of a multi-shot EPI, according to some embodiments of the present disclosure.

FIG. 3A is a 3D visualization of a multi-shot EPI having four shots, according to some embodiments of the present disclosure.

FIG. 3B is a two-dimensional (2D) visualization of a multi-shot EPI having four shots, according to some embodiments of the present disclosure.

FIG. 4A is a 2D table overlaid on k-space indicating an example under-sampling pattern, according to some embodiments of the present disclosure.

FIG. 4B is a 2D table overlaid on k-space indicating an example under-sampling pattern, according to some embodiments of the present disclosure.

FIG. 4C is a 2D table overlaid on k-space indicating an example under-sampling pattern, according to some embodiments of the present disclosure.

FIG. 5 is a flow chart of an exemplary method for generating a 3D EPI image with under-sampling, according to some embodiments of the present disclosure.

FIG. 6A is an example k-space for a 48-shot EPI that is populated with under-sampling, according to some embodiments of the present disclosure.

FIG. 6B is an example k-space for a 48-shot EPI that is populated with under-sampling, according to some embodiments of the present disclosure.

FIG. 6C is an example k-space for a 48-shot EPI that is populated with under-sampling, according to some embodiments of the present disclosure.

FIG. 7A is an example k-space for a 48-shot EPI that is populated with under-sampling, according to some embodiments of the present disclosure.

FIG. 7B is an example k-space for a 48-shot EPI that is populated with under-sampling, according to some embodiments of the present disclosure.

FIG. 8A is a ground truth reconstructed image and three reconstructed images from under-sampling, according to some embodiments of the present disclosure.

FIG. 8B is a ground truth reconstructed image and three reconstructed images from under-sampling, according to some embodiments of the present disclosure.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed.

As used herein, unless otherwise limited or defined, discussion of particular directions is provided by example only, with regard to particular embodiments or relevant illustrations. For example, discussion of “top,” “bottom,” “front,” “rear,” “left,” “right,” “horizontal,” “vertical,” and “longitudinal” features and/or relative motion, e.g., movement “up” and “down,” is generally intended as a description only of the orientation of such features relative to a reference frame of a particular example or illustration. Correspondingly, for example, a “top” feature may sometimes be disposed below a “bottom” feature (and so on), in some arrangements or embodiments. Additionally or alternatively, embodiments may be arranged in a different orientation such that “top” and “bottom” features are arranged horizontally relative to each other, for example in a “left-to-right” orientation.

The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and/or “consisting of” those certain elements.

As stated previously, MRI may be used to obtain internal physiological information about a patient through brain imaging, thoracic imaging, spine imaging, cardiac imaging, and imaging other sections or tissues within a patient's body. More specifically, MRI uses the nuclear magnetic resonance (“NMR”) phenomenon to generate a response in tissue and fluids, and scans this response for signals that are useful for producing images of these tissues and/or fluids. For example, the protons of the atoms in human tissue have relatively small magnetic fields, which may be aligned in random directions. However, when subjected to a uniform magnetic field, such as the so-called main magnetic field (polarizing field B₀) generated by an MRI system, the individual magnetic moments of the nuclei in the tissue attempt to align with this B₀ field. However, the nuclei precess about the axis of the B₀ field, and in random order at their characteristic Larmor frequency. Additionally, when the tissue and fluids are subjected to a magnetic field (excitation field B₁) in the x-y plane and near the Larmor frequency, the net aligned moment, or “longitudinal magnetization,” Mz, may be rotated, or “tipped,” into the x-y plane to produce a net transverse magnetic moment, Mt. The MRI system may then terminate the excitation field B₁. In response, the nuclei emit signals as the nuclei return to their natural, random, magnetic alignment. Accordingly, the MRI system may detect these signals and thus process them to form an image. Further, when using these signals to produce images, the MRI system may employ magnetic field gradients (Gₓ, G_{y}, and G_{z}). Typically, the region to be imaged is scanned in a sequence of measurement cycles during which these gradients, sometimes referred to as readout gradients, vary according to the particular localization method being used. The gradients Gx, Gy, Gz change, e.g., effect, the frequencies and phases of the nuclear moments. The resulting set of received signals are digitized and processed to reconstruct the image using reconstruction techniques.

MRI systems may use specific imaging sequences depending on the type of imaging being performed. The term, imaging sequence, refers to a combination of pulses having specific amplitudes, widths, directions, and time sequences that are applied when a magnetic resonance imaging scan is executed. The pulses may include, as an example, a radio-frequency pulse and gradient pulses. The radio-frequency pulses may include, for example, radio-frequency excitation pulses, radio-frequency refocus pulses, inverse recovery pulses, gradient pulses, and the like. The gradient pulses may include, for example, gradient pulses for slab selection, phase encoding, frequency encoding, phase shifting (phase shift), and dephasing (e.g., the dispersion of phases), and the like.

Echo planar imaging (EPI) is an accelerated imaging technique that can be performed in two dimensions or three. With EPI, an image can be formed from echo signals generated by a single radio-frequency (RF) excitation pulse, also referred to herein as a shot, i.e., a single shot. More specifically, echo planar imaging refers to a sequence in which data from k-space for a 2D plane is collected following a single RF-excitation pulse. However, more recently, the term has expanded to include rapid gradient-echo or spin-echo sequences in which k-space is traversed in one or a small number of excitations, i.e. single or multiple shots. Accordingly, these techniques are referred to as, single shot EPI and multi-shot EPI, respectively.

Three-dimensional (3D) EPI can be useful for detecting various pathologies in the brain. Further, it may be possible to generate relatively high-resolution images for assessing various neurological conditions using T2-star (T2*) and susceptibility-weighted imaging with 3D EPI. For example, relatively high spatial resolution can be useful for visualizing microvascular details, as well as the central vein sign, in white matter multiple sclerosis lesions. In MRI, T2*-weighted imaging uses the T2* relaxation time, which measures how quickly the net magnetization vector decays due to magnetic field inhomogeneities and magnetic susceptibility differences within tissues. These traits make T2*-weighted imaging useful for detecting iron deposition in organs, and detecting microhemorrhages. Additionally, susceptibility-weighted imaging is an MRI sequence that is useful for detecting elements in the blood, such as iron and calcium, because the susceptibility-weighted imaging MRI sequence is sensitive to compounds that distort the local magnetic field.

However, while 3D EPI may provide these useful benefits, 3D EPI may also be time-consuming. As such, under-sampling, also referred to herein as sub-sampling, techniques may be useful for reducing the amount of time for performing 3D EPI scans. Under-sampling involves using a smaller subset of phase encodings in the ky and kz encoding dimensions than a full 3D EPI scan.

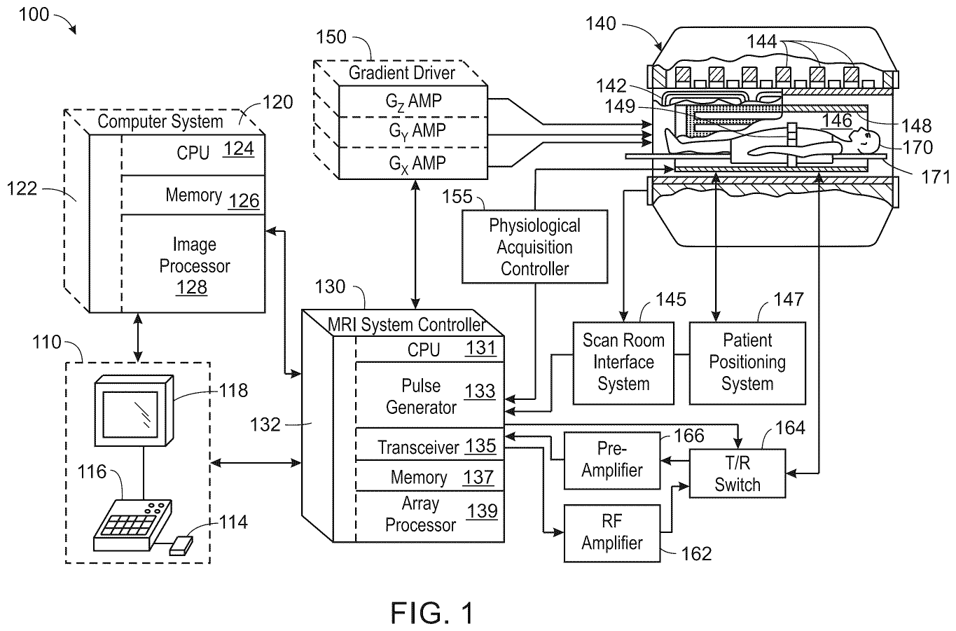

FIG. 1 is a schematic diagram of an MRI system 100 for under-sampling k-space for a three-dimensional (3D) EPI scan performed with magnetic resonance, according to some embodiments of the present disclosure. The operation of MRI system 100 is controlled from an operator workstation 110 that includes an input device 114, a control panel 116, and a display 118. The input device 114 may be a joystick, keyboard, mouse, track ball, touch activated screen, voice control, or any similar or equivalent input device. The control panel 116 may include a keyboard, touch activated screen, voice control, buttons, sliders, or any similar or equivalent control device. The operator workstation 110 is coupled to and communicates with a computer system 120 that enables an operator to control the production and viewing of images on display 118. The computer system 120 includes a plurality of components that communicate with each other via electrical and/or data connections 122. The computer system connections 122 may be direct wired connections, fiber optic connections, wireless communication links, or the like. The components of the computer system 120 include a central processing unit (CPU) 124, a memory 126, which may include a frame buffer for storing image data, and an image processor 128. In an alternative embodiment, the image processor 128 may be replaced by image processing functionality implemented in the CPU 124. The computer system 120 may be connected to archival media devices, permanent or back-up memory storage, or a network. The computer system 120 is coupled to and communicates with a separate MRI system controller 130.

The MRI system controller 130 includes a set of components in communication with each other via electrical and/or data connections 132. The MRI system controller connections 132 may be direct wired connections, fiber optic connections, wireless communication links, or the like. The components of the MRI system controller 130 include a CPU 131, a pulse generator 133, which is coupled to and communicates with the operator workstation 110, a transceiver 135, a memory 137, and an array processor 139. In an alternative embodiment, the pulse generator 133 may be integrated into a resonance assembly 140 of the MRI system 100. The MRI system controller 130 is coupled to and receives commands from the operator workstation 110 to indicate the MR scan sequence to be performed during an MRI scan. The MRI system controller 130 is also coupled to and communicates with a gradient driver system 150, which is coupled to a gradient coil assembly 142 to produce magnetic field gradients during an MR scan. In various embodiments, the components of computer system 120 and MRI system controller 130 may be implemented on the same computer system or a plurality of computer systems.

The pulse generator 133 may also receive data from a physiological acquisition controller 155 that receives signals from a plurality of different sensors connected to an object or patient 170 undergoing an MR scan, including electrocardiography (ECG) signals from electrodes attached to the patient 170. And finally, the pulse generator 133 is coupled to and communicates with a scan room interface system 145, which receives signals from various sensors associated with the condition of the resonance assembly 140. The scan room interface system 145 is also coupled to and communicates with a patient positioning system 147, which sends and receives signals to control movement of a table 171. The table 171 is controllable to move the patient in and out of the core 146 and to move the patient to a desired position within the core 146 for an MR scan.

The MRI system controller 130 provides gradient waveforms to the gradient driver system 150, which includes, among others, G_{X}, G_{Y} and G_{Z} amplifiers. Each G_{X}, G_{Y} and G_{Z} gradient amplifier excites a corresponding gradient coil in the gradient coil assembly 142 to produce magnetic field gradients that spatially encode MR signals during an MR scan. More specifically, the Gx gradient coil assembly 142 encodes the frequencies; and, Gy, Gz encode the phases, of the magnetic field. By acquiring the resultant frequencies and phases, the MRI system controller 130 can determine the spatial location of the water and tissue nuclei in the scanned region of the patient 170. The MRI system controller 130 can make this determination in 2D and 3D Cartesian coordinates, radial coordinates, and/or the like.

The gradient coil assembly 142 is included within the resonance assembly 140, which also includes a superconducting magnet having superconducting coils 144, which in operation, provides a homogenous longitudinal magnetic field B₀ throughout a core 146, or open cylindrical imaging volume, that is enclosed by the resonance assembly 140. The resonance assembly 140 also includes a RF body coil 148 which in operation, provides a transverse magnetic field B₁ that is generally perpendicular to B₀ throughout the core 146. The resonance assembly 140 may also include RF surface coils 149 used for imaging different anatomies of a patient undergoing an MRI scan. The RF body coil 148 and RF surface coils 149 may be configured to operate in a transmit and receive mode, transmit mode, or receive mode.

An object or patient 170 undergoing an MR scan may be positioned within the core 146 of the resonance assembly 140. The transceiver 135 in the MRI system controller 130 produces RF excitation pulses that are amplified by an RF amplifier 162 and provided to the RF body coil 148 and RF surface coils 149 through a transmit/receive switch (T/R switch) 164.

As mentioned above, RF body coil 148 and RF surface coils 149 may be used to transmit RF excitation pulses and/or to receive resulting MR signals from a patient undergoing an MR scan. The resulting MR signals emitted by excited nuclei in the patient undergoing an MR scan may be sensed and received by the RF body coil 148 or RF surface coils 149 and sent back through the T/R switch 164 to a pre-amplifier 166. The amplified MR signals are demodulated, filtered and digitized in the receiver section of the transceiver 135. The T/R switch 164 is controlled by a signal from the pulse generator 133 to electrically connect the RF amplifier 162 to the RF body coil 148 during the transmit mode and connect the pre-amplifier 166 to the RF body coil 148 during the receive mode. The T/R switch 164 may also enable RF surface coils 149 to be used in either the transmit mode or receive mode. The resulting MR signals sensed and received by the RF body coil 148 are digitized by the transceiver 135 and transferred to the memory 137 in the MRI system controller 130.

An MR scan is complete when an array of raw k-space data, corresponding to the received MR signals, has been acquired and stored temporarily in the memory 137 until the data is subsequently transformed to create images. This raw k-space data is rearranged into separate k-space data arrays for each image to be reconstructed, and each of these separate k-space data arrays is input to the array processor 139. Further, the array processor 139 uses a known transformation method, most commonly a Fourier transform, to create images from the received MR signals. These images are communicated to the computer system 120 where they are stored in memory 126. In response to commands received from the operator workstation 110, the image data may be archived in long-term storage or it may be further processed by the image processor 128 and conveyed to the operator workstation 110 for presentation on the display 118.

According to some embodiments of the present disclosure, the MRI system controller 130 may make it possible to generate 3D EPI imaging by under-sampling the MR signals in the ky phase and slab (kz phase) encodings. Slab encoding is also a phase encoding, but along a different axis (i.e., the kz axis) than the ky phase encoding. More specifically, the MRI system controller 130 may under-sample (i.e., sub-sample) ky phase encoding using a coherent pattern. Additionally, the MRI system controller 130 may sub-sample slab encodings using an incoherent pattern for each ky phase encoding. Coherent patterns may follow uniform, e.g., constant, distances in k-space. Conversely, incoherent patterns may indicate non-uniform, e.g., random, distances in k-space.

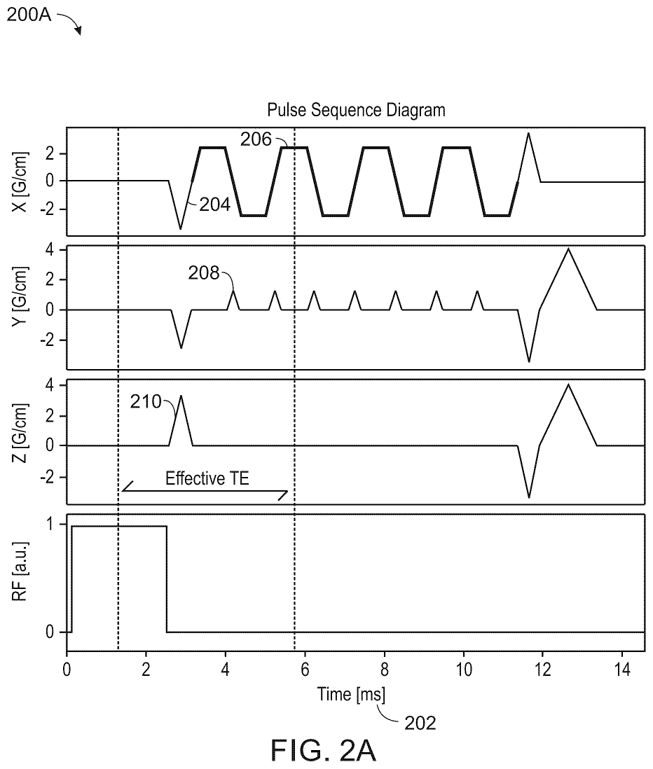

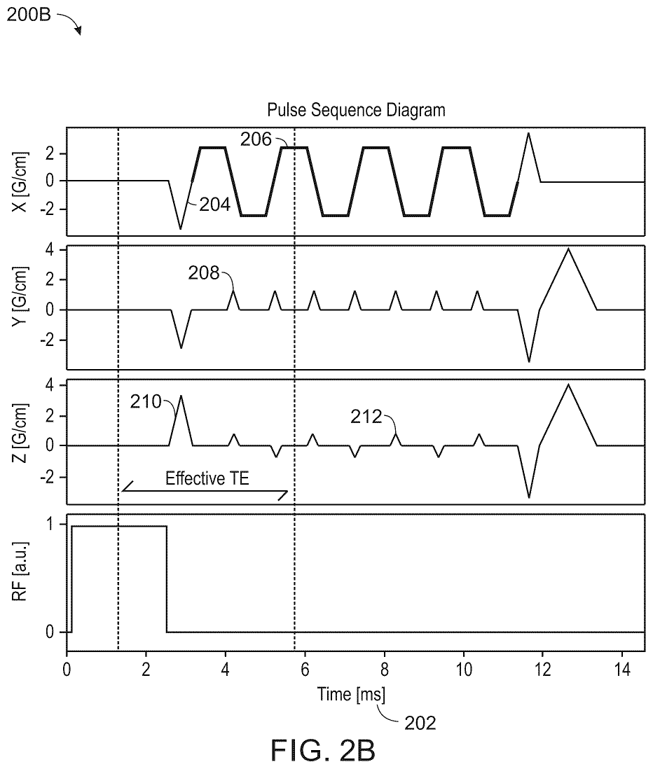

FIG. 2A is a pulse sequence diagram 200A representing one shot of a multi-shot EPI, according to some embodiments of the present disclosure. The pulse sequence diagram 200A includes a series of signals over time 202. Collectively, this series of signals may be referred to as a readout train. As indicated, the signals include: “X,” i.e., the frequency (kx) encoding signal; “Y,” the ky phase encoding signal; “Z,” the kz phase signal; and, “RF,” the radio frequency excitation signal. The frequency encoding signal includes a series of readout lobes 204, each having an upper plateau 206. The MRI system controller 130 acquires (e.g., reads out) the MR echo signal (not shown) at a time approaching and following the midpoint of the upper plateaus 206. The line labeled, “Effective TE,” indicates the effective time to echo, i.e., the time from the center of the RF excitation pulse to the k-space center traverse in ky. The ky phase encoding signal includes a series of ky phase encoding gradient blips 208, which occur between the readout lobes 204. The ky phase encoding gradient blips 208 cause each MR echo signal to be separately ky phase encoded at each readout (i.e., signal acquisition). According to some embodiments of the present disclosure, the ky phase encoding gradient blips 208 occupy the same area, and are separated by a constant time, thus providing a constant time difference between echoes, and a constant distance between phase encodings in the ky dimension. In this way, the ky phase encoding gradient blips 208 may enable the acquisition (e.g., readout) of a coherent sampling pattern in the ky dimension. Additionally, the kz phase encoding signal includes one kz dephaser 210. The kz dephaser 210 may cause the MR echo signals to be kz phase encoded for one specific kz phase, e.g., the phase coordinate in the kz dimension. Thus, to enable the acquisition of an incoherent sub-sampling pattern in the kz dimension, the MRI system controller 130 may change the phase of the kz dephaser for each RF excitation pulse. For example, for each RF excitation pulse, the MRI system controller 130 may select a different kz phase based on a pseudo-random pattern. In this way, a multi-shot EPI according to some embodiments of the present disclosure may provide a different kz phase encoding for each RF excitation pulse. Thus, the multiple, non-uniform (e.g., pseudo-random), kz phase encodings generated for multiple RF excitations pulses may, collectively, enable the acquisition of an incoherent sub-sampling pattern in the kz dimension for a multi-shot EPI.

FIG. 2B is a pulse sequence diagram 200B representing one shot of a multi-shot EPI, according to some embodiments of the present disclosure. The pulse sequence diagram 200B is similar to the pulse sequence diagram 200A, having signals, “X,” “Y,” and, “Z,” over time 202; and, “RF,” the radio frequency excitation signal. In this example, the X, Y, and RF signals are similar to those of the pulse sequence diagram 200A. However, in contrast to the pulse sequence diagram 200A, the pulse sequence diagram 200B additionally includes a series of kz phase encoding gradient blips 212 for the Z signal. Similar to the ky phase encoding gradient blips, the kz phase encoding gradient blips 212 occur between the readout lobes 204, and cause each MR echo signal to be separately phase encoded (although in the kz dimension). In contrast to the ky phase encoding gradient blips 208 though, the kz phase encoding gradient blips 212 are not constant, e.g., separated by irregular distances in time. Accordingly, MR signals encoded with the kz phase encoding gradient blips 212 have an irregular distance between phase encodings in the kz dimension. As such, the kz phase encoding gradient blips 212 cause small changes of kz phase within one RF excitation pulse. In this way, some embodiments of the present disclosure may increase the incoherence of the sub-sampling pattern in the kz dimension, which may increase the sharpness of the image.

If the size of the phase encoding gradient blips 208, 212 between readout lobes 206 is not constant, under-sampling may produce additional geometrical distortions and susceptibility artifacts. However, for higher acceleration factors in the ky and kz phase encoding dimensions, an irregular (i.e., incoherent) under-sampling pattern may reduce aliasing errors and provide sharper, and thus more useful, images. As such, providing useful images for 3D EPI scans using conventional under-sampling techniques can be challenging.

The echo time refers to the time from the center of the RF excitation pulse to the time of signal acquisition (e.g., the center of the readout lobes 206). Thus, since in EPI several lines (e.g., ky and kz phase encodings) are acquired per RF excitation pulse, the echo time of each phase encoding may not be constant. Rather, in EPI, the echo time of each phase encoding may increase linearly with its index in k-space because the MR signals resulting from the phase encodings are acquired one after the other. Further, the area of the gradient blips is constant across the readout train. This property is also referred to as coherent sampling in k-space. However, with this approach, EPI can produce geometric distortions, while also providing relatively sharp point spread function. Hence, despite the system imperfections resulting from off-resonance signals or the like, EPI may still create coherent artifacts with coherent sampling. More specifically, while such distortions may move features (i.e., put the features at the wrong location) in the image, EPI may still provide relatively sharp resolution of these features.

The inventors have recognized that improved under-sampling techniques of 3D EPI may be useful. Accordingly, some embodiments of the present disclosure may provide an under-sampling technique with coherent sub-sampling in the ky phase encoding dimension, and incoherent sampling in the kz phase encoding dimension. Further, such embodiments may use a deep learning model to reconstruct the images from this under-sampling. In these ways, such embodiments may reduce the length of time that patients undergoing 3D EPI imaging may spend undergoing an MRI scan, while maintaining the quality, and thus, the usefulness of the images generated.

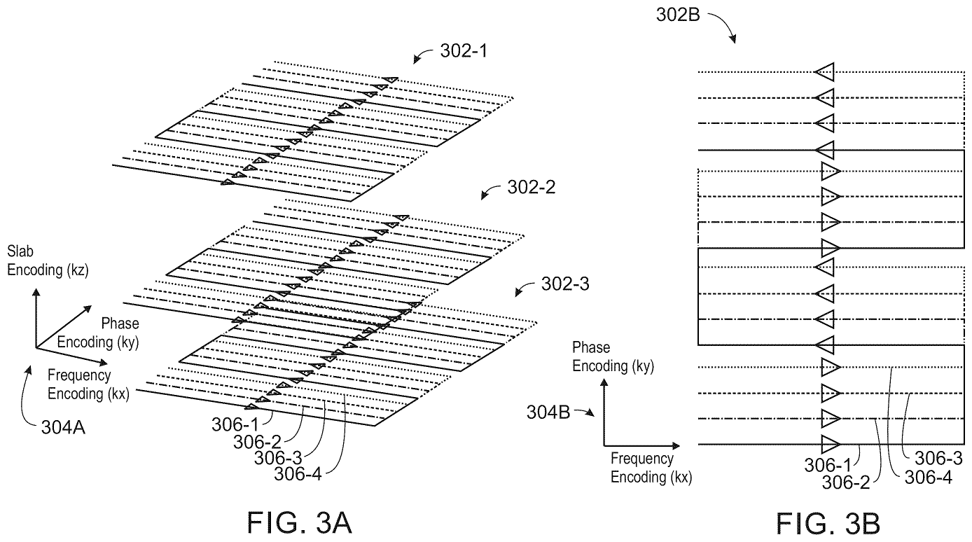

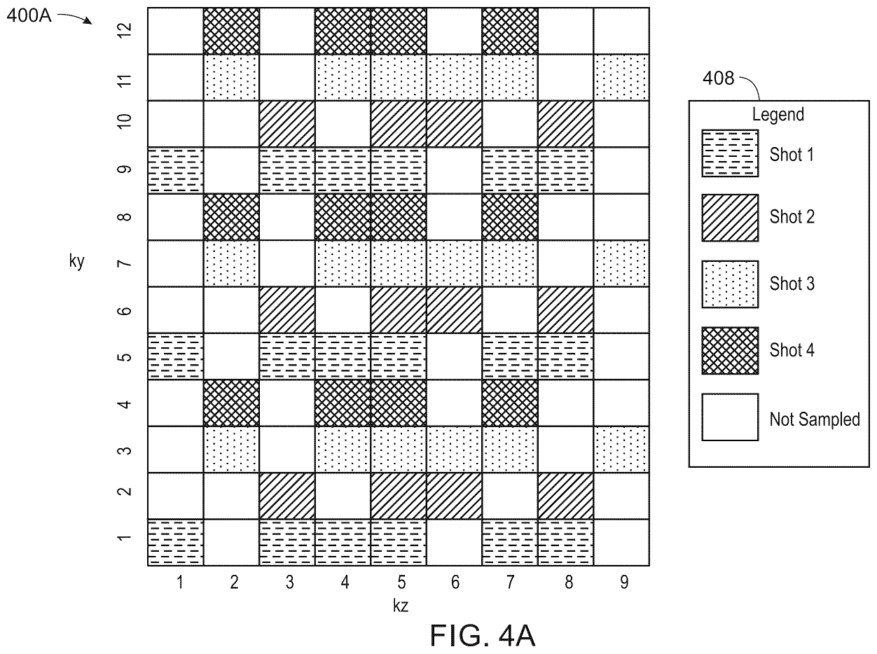

FIG. 3A is a 3D visualization of a multi-shot EPI having four shots, according to some embodiments of the present disclosure. This multi-shot EPI is represented in three planes 302-1, 302-2, 302-3 (collectively referred to as planes 302), in the ky-kx dimensions, and separated in the kz dimension, as indicated in the 3D k-space legend 304A. Additionally, each of the lines, referred to herein as shots 306-1, 306-2, 306-3, 306-4 (collectively referred to as shots 306) represent different shots of an MRI scan, and the ky phase encodings that are sub-sampled for the respective shots 306. Further, each plane 302 represents a slab (kz phase) encoding that is sampled. The planes 302 indicate the trajectory of phase and frequency encodings in all three dimensions of 3D k-space including the kx direction. As understood by one of ordinary skill, kx is the readout dimension, which is not subsampled. Rather, the MRI system controller 130 fully samples the kx frequency encodings. Thus, for one RF excitation pulse, the MRI system controller 130 may acquire multiple kx lines, where each kx line represents one ky phase encoding within a shot 306. Thus, for one RF excitation pulse, the MRI system controller 130 may acquire several of those kx frequency lines. As such, each kx lines may be referred to by the index pair [ky, kz],

FIG. 3B is a two-dimensional (2D) visualization of a multi-shot EPI having four shots, according to some embodiments of the present disclosure. This multi-shot EPI is represented in one ky-kx plane 302B. As indicated in the 2D k-space legend 304B, the ky-kx plane 302B represents the ky phase encoding and frequency encoding (kx) dimensions. As stated previously, each of the shots 306-1, 306-2, 306-3, 306-4 represent different shots of an MRI scan, and trace a coherent pattern along the phase encodings that are sub-sampled for each of the shots, one through four. In this way, the ky-kx plane 302B represents a coherent sub-sampling pattern that is based on the total number of shots. In this example, the plane 302B indicates the trajectory of ky phase and frequency encodings in the ky and kx dimensions, respectively. Thus, each of the shots 306-1, 306-2, 306-3, 306-4 includes multiple kx lines that are acquired according to the techniques described herein.

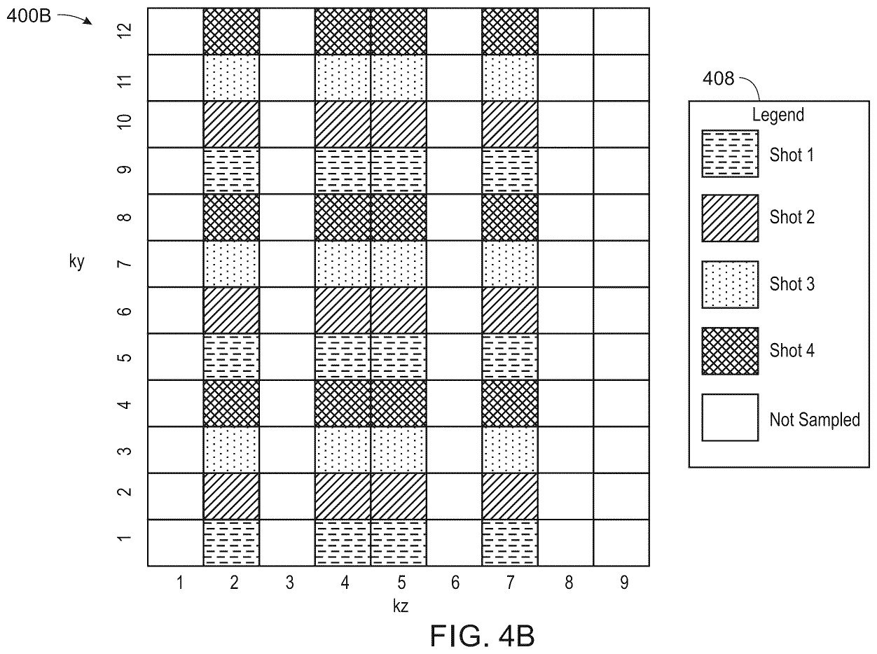

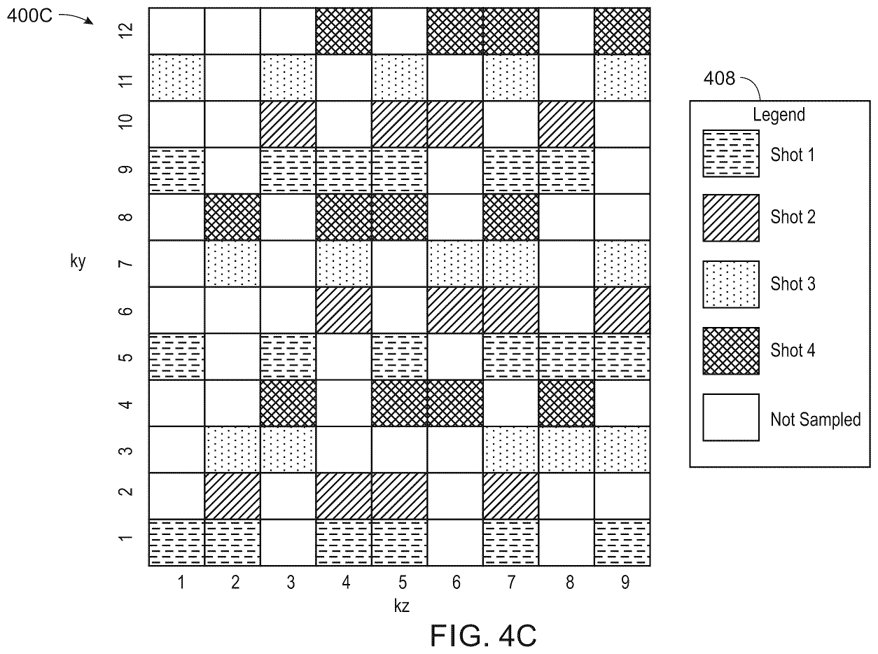

FIG. 4A is a 2D table 400A overlaid on k-space indicating an example under-sampling pattern, according to some embodiments of the present disclosure. The two dimensions of table 400A include the ky and kz phase encodings. Further, the table 400A includes values ranging from 1 to 12 for the ky phase encodings. Additionally, the table 400A includes values ranging from 1 to 9 for the kz phase encodings. These values are indicated for clarity of description, and do not represent actual ky and kz phase encoding values. Further, the legend 408 includes hatch patterns indicated in the cells of table 400A, where each hatch pattern indicates a specific shot for sampled ky and kz phase encoding combinations, and blanks indicate ky and kz phase encoding combinations that are not. As stated previously, the MRI system controller 130 acquires multiple lines of kx frequency encodings for each shot. As such, each kx frequency line may be referred to by the index pair [ky, kz]. Accordingly, each cell of the table 400A represents one line of sampled kx frequency encodings.

In table 400A, the sub-sampling of the ky phase encodings is coherent, and based on the total number of shots. Additionally, the sub-sampling of the kz phase encodings is incoherent, and, in this example, varies by shot. Hence, for shot 1 of a four-shot EPI, the MRI system controller 130 may sub-sample ky phase encodings 1, 5, and 9; specifically, kz phase encodings 1, 3-5, 7, and 8, for each of these ky phase encodings. For shot 2, the MRI system controller 130 may sample ky phase encodings 2, 6, and 10; specifically, kz phase encodings 3, 5, 6, and 8, for each of these ky phase encodings. For shot 3, the MRI system controller 130 may sample ky phase encodings 3, 7, and 11; specifically, kz phase encodings 2, 4-7, and 9, for each of these ky phase encodings. For shot 4, the MRI system controller 130 may sample ky phase encodings 4, 8, and 12; specifically, kz phase encodings 2, 4, 5, and 7, for each of these ky phase encodings.

Table 400A represents a specific example of coherently sub-sampled ky phase encodings and incoherently sub-sampled kz phase encodings. More generally, the MRI system controller 130 may sub-sample phase and frequency encodings in terms of: ky and kz phases, kx frequencies, and Ns shots. For example, suppose k_{y}=1, . . . , N_{y}, and k_{z}=1, . . . , N_{z}. Suppose Nₛ denotes the number of shots. “Shot 1” samples k_{y}∈S₁={1≤k_{y}≤N_{y}:k_{y}=1,1+Nₛ, 1+2Nₛ, . . . }, “Shot 2” samples k_{y}∈S₂={1≤k_{y}≤N_{y}:k_{y}=2,2+Nₛ, 2+2Nₛ, . . . }, . . . , and “Shot Nₛ” samples k_{y}∈S_{N}_{ₛ}={1≤k_{y}≤N_{y}:k_{y}=Nₛ, 2Nₛ, 3Nₛ, . . . }. For each k_{z}, a subset Mₖ_{_{z}}⊂{1, . . . , Nₛ} is selected such that Shot m∈Mₖ_{_{z}} samples k_{y}∈Sₘ, and Shot n∉Mₖ_{_{z}} is skipped. A constraint here is that the set Sₘ of k_{y}'s sampled in Shot m is based on regular sampling with period Nₛ, the number of shots. For each k_{z}, k_{y}∈¿m∈Mₖ_{_{z}} Sₘ are sampled where Mₖ_{_{z}} is a subset of {1, . . . , Nₛ}. In other words, the constraint can be written as follows. If (k_{y}, k_{z}) is sampled, then (k_{y}±Nₛ, k_{z}), (k_{y}±2Nₛ, k_{z}), (k_{y}±3Nₛ, k_{z}), . . . are also sampled (within legitimate ranges).

As stated previously, the MRI system controller 130 may select the kz phase encodings to generate an incoherent sub-sampling pattern. According to some embodiments of the present disclosure, incoherent sub-sampling may be random, pseudo-random, and the like. Pseudo-random selections may be based on a variable density Poisson disk (VDPD), or other patterns in one (e.g, the kz phase encoding) or two (e.g., the ky and kz phase encoding) dimensions. In this way, such embodiments may reduce clusters in k-space of sampled ky and kz phase encodings.

FIG. 4B is a 2D table 400B overlaid on k-space indicating an example under-sampling pattern, according to some embodiments of the present disclosure. The table 400B is similar to the table 400A, and represents a coherent sub-sampling in the ky phase encoding and an incoherent sampling pattern in the kz phase encoding. Further, each [ky, kz] cell of the table 400B also represents one line of sampled kx frequency encodings. However, in contrast to table 400A, the table 400B includes the same incoherent kz phase sub-sampling pattern for each ky phase encoding, and for each shot. Hence, for shots 1-4 of a four-shot EPI, the MRI system controller 130 may sub-sample kz phase encodings 2, 4, 5, and 7, for each of shot 1 (ky phase encodings 1, 5, 9); shot 2 (ky phase encodings 2, 6, and 10); shot 3 (ky phase encodings 3, 7, and 11); and shot 4 (ky phase encodings 4, 8, and 12).

FIG. 4C is a 2D table 400C overlaid on k-space indicating an example under-sampling pattern, according to some embodiments of the present disclosure. The table 400C is similar to the table 400A and represents a coherent sub-sampling in the ky phase encoding and an incoherent sampling pattern in the kz phase encoding. Additionally, each [ky, kz] cell of the table 400C also represents one line of sampled kx frequency encodings. Further, for each shot, the table 400C indicates the same number of sub-sampled slab encodings for each ky phase encoding in a shot. However, in contrast to the table 400A, the table 400C includes a different set of sub-sampled slab encodings for each ky phase encoding in the shot. Hence, for shot 1 of a four-shot EPI, the MRI system controller 130 may sub-sample ky phase encodings 1, 5, and 9, with different sets of six slab encodings for each ky phase encoding. More specifically, for ky phase encoding 1, slab encodings 1, 2, 4, 5, 7, and 9 are sub-sampled. For ky phase encoding 5, slab encodings 1, 3, 5, and 7-9 are sub-sampled. For ky phase encoding 9, slab encodings 1, 3-5, 7, and 8 are sub-sampled.

For shot 2, the MRI system controller 130 may sub-sample ky phase encodings 2, 6, and 10, with different sets of four slab encodings for each ky phase encoding. More specifically, for ky phase encoding 2, slab encodings 2, 4, 5, and 7 are sub-sampled. For ky phase encoding 6, slab encodings 4, 6, 7, and 9 are sub-sampled. For ky phase encoding 10, slab encodings 3, 5, 6, and 8 are sub-sampled.

For shot 3, the MRI system controller 130 may sub-sample ky phase encodings 3, 7, and 11, with different sets of five slab encodings for each ky phase encoding. More specifically, for ky phase encoding 3, slab encodings 2, 3, and 7-9 are sub-sampled. For ky phase encoding 7, slab encodings 2, 4, 6, 7, and 9 are sub-sampled. For ky phase encoding 11, slab encodings 1, 3, 5, 7, and 9 are sub-sampled.

For shot 4, the MRI system controller 130 may sub-sample ky phase encodings 4, 8, and 12, with different sets of four slab encodings for each ky phase encoding. More specifically, for ky phase encoding 4, slab encodings 3, 5, 6, and 8 are sub-sampled. For ky phase encoding 8, slab encodings 2, 4, 5, and 7 are sub-sampled. For ky phase encoding 12, slab encodings 4, 6, 7, and 9 are sub-sampled.

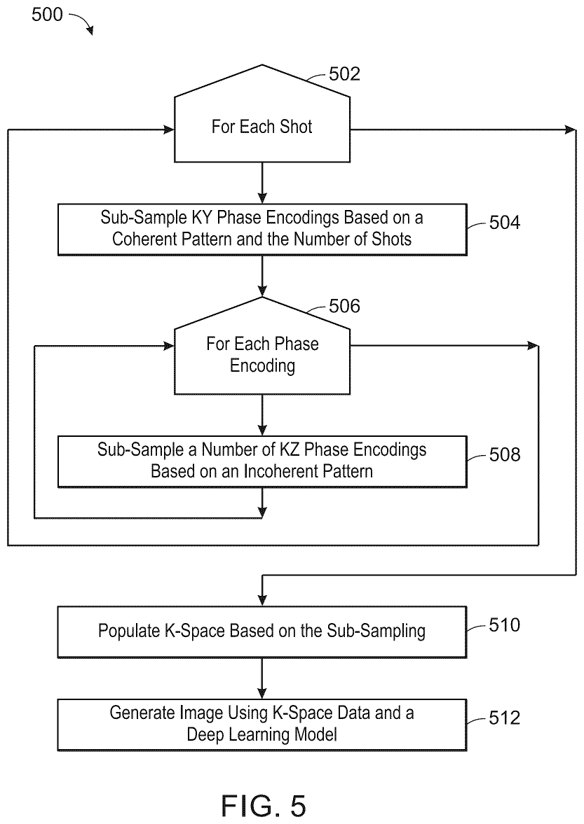

FIG. 5 is a flow chart of an exemplary method 500 for generating a 3D EPI image with under-sampling, according to some embodiments of the present disclosure. The MRI system controller 130, described with respect to FIG. 1, may perform the method 500. More specifically, the MRI system controller 130 may perform operations 502 through 512 for each shot of a mult-shot 3D EPI. As stated previously, a shot is an RF excitation pulse.

At operation 504, the MRI system controller 130 may sub-sample ky phase encodings for the shot based on a coherent sub-sampling pattern and the number of shots. As stated previously, coherent sub-sampling of the ky phase encodings may include generating ky phase encoding gradient blips 208 having a constant size in area, and acquiring the resultant phase encoded signals. Additionally, after each ky phase encoding gradient blip 208, the MRI system controller 130 may acquire a line of kx frequency encodings. Further, the coherent sub-sampling pattern may be based on the number of shots in the MRI scan. Hence, in a 48-shot MRI scan, the MRI system controller 130 may sub-sample ky phase encodings 1, 49, and 97 for shot 1; ky phase encodings 2, 50, and 98 for shot 2; ky phase encodings 3, 51, and 99 for shot 3; and, the like. Additionally, the MRI system controller 130 may acquire the ky phase encoded MR echo signals. Further, the MRI system controller 130 may perform operations 506 through 512 for each ky phase encoding in the shot.

At operation 508, the MRI system controller 130 may sub-sample a number of kz phase encodings based on an incoherent pattern. As stated previously, the MRI system controller 130 may generate a kz dephaser 210 that enables the acquisition of the incoherent sub-sampling pattern in the kz dimension. More specifically, the kz dephaser 210 may, for each shot of a multi-shot EPI, provide a kz phase encoding gradient with a pseudo-randomly selected kz phase encoding. Further, the MRI system controller 130 may acquire the kz phase encoded MR echo signals Additionally, according to some embodiments of the present disclosure, the MRI system controller 130 may generate one or more kz phase encoding gradient blips 212 having irregular size in area, to increase the incoherence of the sub-sampling pattern.. The incoherent sub-sampling pattern may be random, pseudo-random, and the like. According to some embodiments of the present disclosure, the incoherent sub-sampling pattern may be based on a 1-dimensional VDPD in the kz dimension. Alternatively, the incoherent sub-sampling pattern may be based on a 2-dimensional VDPD in the kz and ky dimensions. In such embodiments, the MRI system controller 130 may use a different random seed for selecting the kz phase encodings to sample. In these ways, the MRI system controller 130 may mitigate the density in k-space of clusters of the sub-sampled ky and kz phase encodings. According to some embodiments of the present disclosure, the incoherent sub-sampling pattern may be based on a 2D VDPD in at least a portion of the ky-kz space. The 2D VDPD sampling pattern in the portion of the ky-kz space may be used for determining a sampling pattern for the remaining portion of the ky-kz space. For example, in table 400A, described with respect to FIG. 4A, a portion of the ky-kz space corresponding to ky=1-4 (where 4 is the number of shots) and kz=1-9 may be determined based on a 2D VDPD sampling by using pseudo-random number generation or a deterministic density function, and this portion may be repeated for ky=5-8 and kz=1-9 and for ky=9-12 and kz=1-9.

As stated previously, the MRI system controller 130 may sub-sample the same number of kz phase encodings for each ky phase encoding of a shot. Further, in some embodiments of the present disclosure, the MRI system controller 130 may sub-sample the same kz phase encodings for each ky phase encoding, and for each shot, as indicated in table 400B. Alternatively, the MRI system controller 130 may sub-sample the same specific kz phase encodings for each ky phase encoding in a shot, as indicated in table 400A. Further, according to some embodiments of the present disclosure, the MRI system controller 130 may sub-sample a different set of kz phase encodings for each of the ky phase encodings in a shot, as indicated in table 400C. According to some embodiments of the present disclosure, the sub-sampling may be based on a 2D VDPD sampling in at least a portion of the ky-kz space. For example, in table 400C, described with respect to FIG. 4C, each of three portions of the ky-kz space, defined by ky=1-4, ky=5-8 and ky=9-12, may be sub-sampled independently by 2D VDPD sampling, with constraints that the numbers of sampled kz encoding values are the same for ky=1, 5, 9; for ky=2, 6, 10; for ky=3, 7, 11; and ky=4, 8, 12.

At operation 510, the MRI system controller 130 may populate a k-space based on the sub-sampling. In other words, the MRI system controller 130 may populate k-space with the sub-sampled ky and kz phase encodings.

At operation 512, the image processor 128 may generate an image using the data in the populated k-space and a deep learning model. According to some embodiments of the present disclosure, the deep learning model may include a densely connected iterative deep learning network.

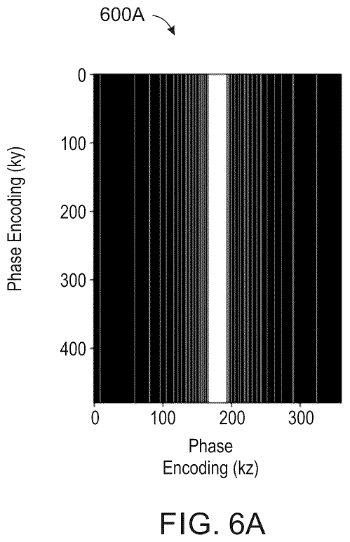

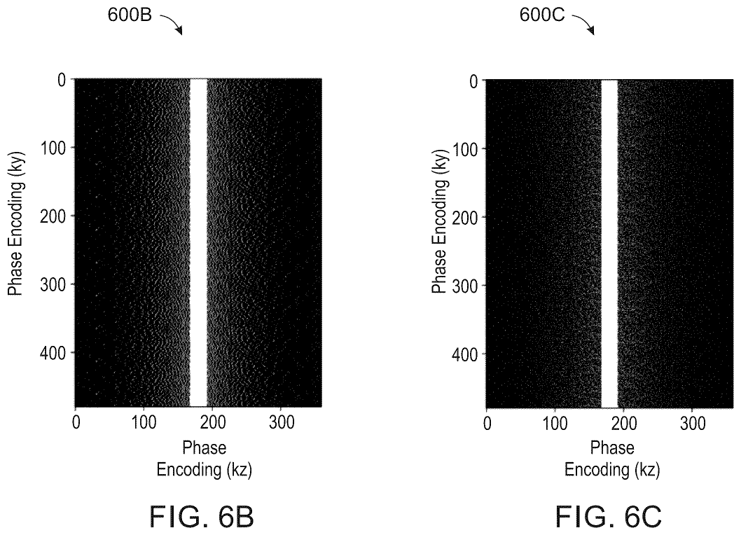

FIG. 6A is an example k-space 600A for a 48-shot EPI that is populated with under-sampling, according to some embodiments of the present disclosure. The k-space 600A includes ky and kz axes of ky and kz phase encodings, respectively. In this example, the ky phase encodings range from 0 to 480, and the kz phase encodings range from 0 to 360. As stated previously, the MRI system controller 130 samples the same number of kz phase encodings for each ky phase encoding in a shot. Further, the MRI system controller 130 may select the kz phase encodings using an incoherent sub-sampling pattern. In this example, the MRI system controller 130 makes a pseudo-random selection of kz phase encodings using a one-dimensional (1D) VDPD pattern in the kz dimension. Additionally, the MRI system controller 130 uses the same kz phase encodings for each ky phase encoding, and for each shot. As such, the k-space 600A indicates the same incoherent sub-sampling pattern of kz phase encoding for all the ky phase encodings in the k-space 600A. In this way, the k-space 600A represents a sub-sampling pattern similar to that of table 400B.

FIG. 6B is an example k-space 600B for a 48-shot EPI that is populated with under-sampling, according to some embodiments of the present disclosure. The k-space 600B is similar to the k-space 600A, including ky and kz axes of ky phase encodings and kz phase encodings, respectively. In this example, the MRI system controller 130 generates an incoherent sub-sampling pattern in the kz dimension with a pseudo-random selection of kz phase encodings using a 1D VDPD pattern for each shot plus a fully-sampled center region, also known as an auto-calibration signal (ACS). Accordingly, the MRI system controller 130 sub-samples the same kz encodings for all the ky phase encodings of a shot. As such the k-space 600B indicates the same sampling pattern for the ky phase encodings of a shot. Hence, phase encoding 1 has the same kz phase encodings (e.g., sub-sampling pattern) as phase encoding 49, 97, and so on. Similarly, phase encoding 2 has a different set of kz phase encodings than phase encoding 1, but the same kz phase encodings as phase encoding gradients 50, 98, and so on. In this way, the k-space 600B represents a sub-sampling pattern similar to that of table 400A.

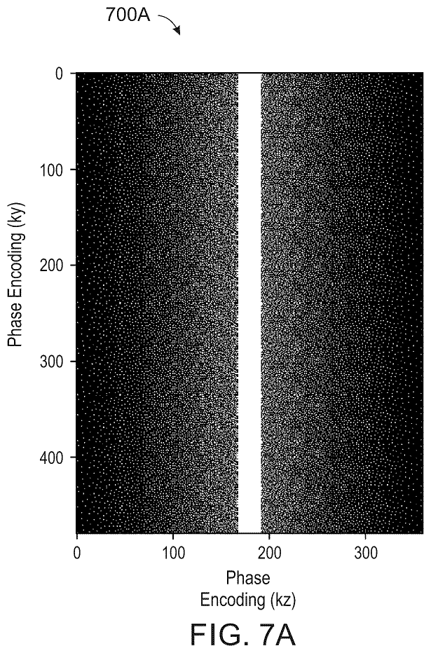

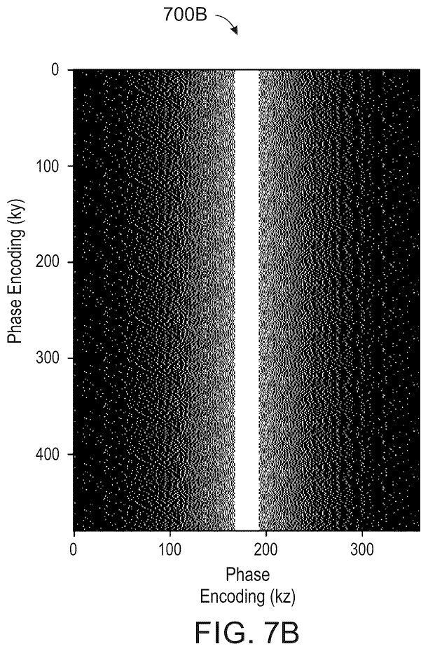

FIG. 6C is an example k-space 600C for a 48-shot EPI that is populated with under-sampling, according to some embodiments of the present disclosure. The k-space 600C is similar to k-space 600A, including ky and kz axes of ky and kz phase encodings, respectively. In this example, the MRI system controller 130 populates k-space 600C with an incoherent sub-sampling pattern in the kz dimension by selecting a pseudo-random set of kz phase encodings by using a 2D VDPD pattern for each shot. Accordingly, the MRI system controller 130 selects the kz phase encodings for a ky phase encoding based on a predetermined density function and the neighboring samples in both the ky and kz phase encodings. For example, in FIG. 7A, the density selection may be a function that includes fully sampled data for the middle kz phase encodings, with a decreasing density in kz towards the outer k-space. Additionally, the density ky may reduce as a function of kz. According to some embodiments of the present disclosure, a gaussian function, or the like may be used to determine this and other like density definitions. By using a 2D VDPD sampling pattern, embodiments of the present disclosure may reduce the clustering of samples in both the ky and kz dimensions of k-space 600C. In this way, the k-space 600A represents a sub-sampling pattern similar to that of table 400C.

FIG. 7A is an example k-space 700A for a 48-shot EPI that is populated with under-sampling, according to some embodiments of the present disclosure. The k-space 700A includes ky and kz axes of ky and kz phase encodings, respectively. The ky phase encodings range from 0 to 480, and the kz phase encodings range from 0 to 360. In this example, the MRI system controller 130 generates an incoherent sub-sampling pattern using a 2D VDPD pattern to select kz phase encodings for each shot. Accordingly, the MRI system controller 130 pseudo-randomly selects the kz phase encodings for the phase encodings of the shot based on a predetermined density selection and the neighboring samples in both the ky and kz dimensions. By using a 2D VDPD sampling pattern, embodiments of the present disclosure may reduce the clustering of samples in both the ky and kz dimensions of k-space 700A. In this way, some embodiments of the present disclosure may provide a coherent sub-sampling of ky phase encodings, and an incoherent sub-sampling pattern of kz phase encodings. Populating k-space in this way may be useful for reconstructing sharper images with reduced aliasing errors in comparison to current techniques.

FIG. 7B is an example k-space 700B for a 48-shot EPI that is populated with under-sampling, according to some embodiments of the present disclosure. The k-space 700B is similar to k-space 700A, including ky and kz axes of respective ky and kz phase encodings. In this example, the MRI system controller 130 makes a random selection of kz phase encodings using a 1D VDPD pattern for each shot. Accordingly, the MRI system controller 130 uses the same kz phase encodings for all the ky phase encodings of a shot. Hence, ky phase encoding 1 has the same set of sub-sampled kz phase encodings as ky phase encodings 49, 97, and so on. Similarly, phase encoding 2 has a different set of sub-sampled kz phase encodings from ky phase encoding 1, but the same kz phase encodings as phase encodings 50, 98, and so on. In this way, some embodiments of the present disclosure may provide a coherent sub-sampling of ky phase encodings, and an incoherent sub-sampling pattern of kz phase encodings. Populating k-space in this way may be useful for reconstructing sharper images with reduced aliasing errors in comparison to current techniques.

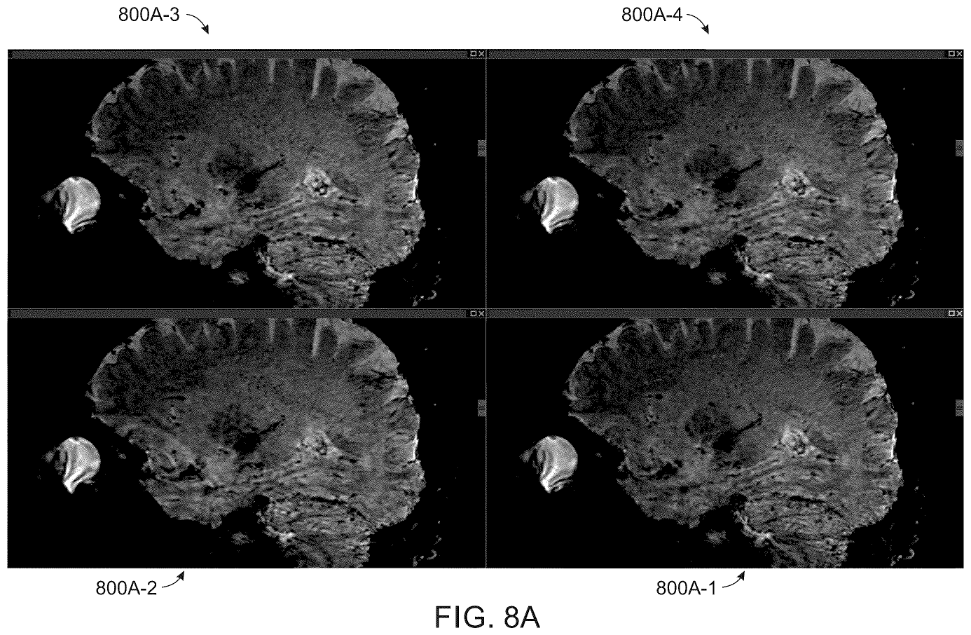

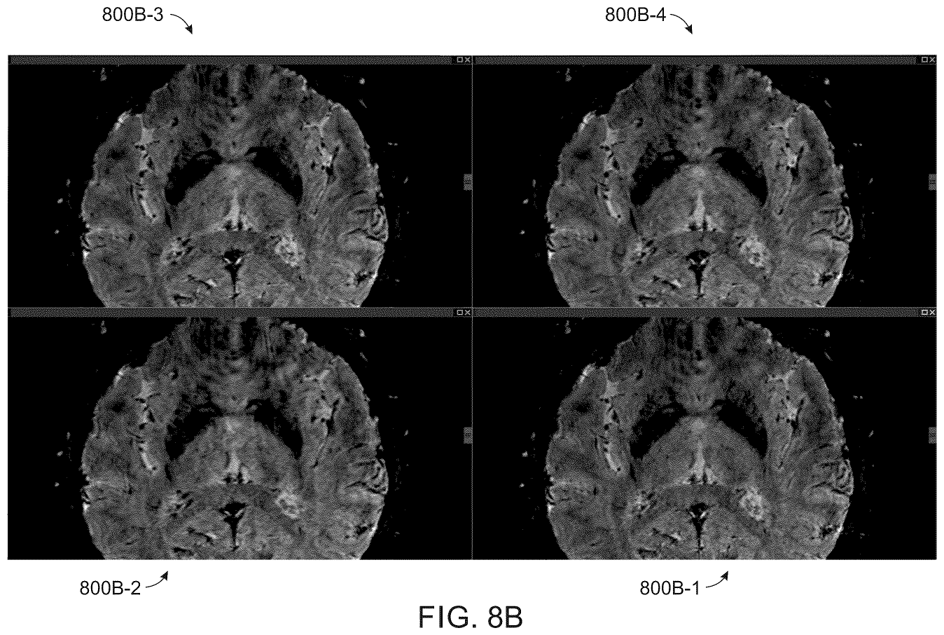

FIG. 8A is a ground truth reconstructed image 800A-1 and three reconstructed images 800A-2, 800A-3, 800A-4 from under-sampling, according to some embodiments of the present disclosure. The ground truth reconstructed image 800A-1 is reconstructed using a full sampling. A full sampling involves a scan of all ky phase encodings and kz phase encodings within the predetermined k-space dimensions. However, a scan with full sampling takes a longer time to perform than the under-sampling techniques described herein. Longer scans can be problematic for patients who suffer from claustrophobic conditions. Additionally, longer scans increase the amount of time within which a patient may move, potentially introducing errors into the k-space data, which may reduce the accuracy of the scan. Advantageously, under-sampling as described herein, may reduce the time involved in performing the scan, thus, reducing patient discomfort, and the potential for scanning errors from patient movement.

In contrast to the ground truth reconstructed image 800A-1, the reconstructed image 800A-2 may be reconstructed using a deep learning network, and under-sampled k-space data according to some embodiments of the present disclosure. More specifically, the under-sampled k-space data for the reconstructed image 800A-2 may be generated by using the 1D VDPD under-sampling pattern indicated in k-space 600A, as described with respect to FIG. 6A. As stated previously, the k-space 600A is populated using the same kz phase encodings for each ky phase encoding, and for each shot.

Additionally, the reconstructed image 800A-3 may be reconstructed using a deep learning network, and under-sampled k-space data according to some embodiments of the present disclosure. More specifically, the under-sampled k-space data for the reconstructed image 800A-3 may be generated by using the 2D VDPD under-sampling pattern indicated in k-space 600C, as described with respect to FIG. 6C. As stated previously, the k-space 600C may mitigate the clustering of samples in both the ky and kz dimensions of k-space 600C. Further, the k-space 600C is populated using the same number of, but a different set of, kz phase encodings for each ky phase encoding of a shot.

Additionally, the reconstructed image 800A-4 may be reconstructed using a deep learning network, and under-sampled k-space data according to some embodiments of the present disclosure. More specifically, the under-sampled k-space data for the reconstructed image 800A-4 may be generated by using the 1D VDPD under-sampling pattern indicated in k-space 600B, as described with respect to FIG. 6B. As stated previously, the k-space 600B is populated using the same kz phase encodings for each phase encoding of a shot.

FIG. 8B is a ground truth reconstructed image 800B-1 and three reconstructed images 800B-2, 800B-3, 800B-4, according to some embodiments of the present disclosure. The reconstructed images 800B-1, 800B-2, 800B-3, 800B-4 represent images of the same scanned region shown in reconstructed images 800A-1, 800A-2, 800A-3, 800A-4, but from a top perspective. Accordingly, the ground truth reconstructed image 800B-1 is reconstructed using a full sampling.

In contrast to the ground truth reconstructed image 800B-1, the reconstructed image 800B-2 may be reconstructed using the k-space data generated by using the 1D VDPD under-sampling pattern indicated in k-space 600A, as described with respect to FIG. 6A. As stated previously, the k-space 600A is populated using the same kz phase encodings for each ky phase encoding, and for each shot.

Additionally, the reconstructed image 800B-3 may be reconstructed using the k-space data generated with the 2D VDPD under-sampling pattern indicated in k-space 600C, as described with respect to FIG. 6C. As stated previously, the k-space 600C may reduce the clustering of samples in both the ky and kz dimensions of k-space 600C. Further, the k-space 600C is populated using the same number of, but a different set of, kz phase encodings for each ky phase encoding of a shot.

Further, the reconstructed image 800B-4 may be reconstructed using the k-space data generated by using the 1D VDPD under-sampling pattern indicated in k-space 600B, as described with respect to FIG. 6B. As stated previously, the k-space 600B is populated using the same kz phase encodings for each ky phase encoding of a shot.

In various embodiments, any suitable computer-readable media can be used for storing instructions for performing functions and/or processes described herein. For example, in some embodiments, computer-readable media can be transitory or non-transitory. For example, non-transitory computer-readable media can include media such as magnetic media (such as hard disks, floppy disks, etc.), optical media (such as compact discs, digital video discs, Blu-ray discs, etc.), semiconductor media (such as RAM, Flash memory, electrically programmable read-only memory (EPROM), electrically erasable programmable read-only memory (EEPROM), etc.), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

This written description uses examples to disclose the invention(s), including the best mode, and also to enable any person skilled in the art to make and use the invention(s). Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention(s) is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

1. A method of sub-sampling a k-space for a magnetic resonance imaging (MRI) scan, comprising:
for each radio-frequency excitation pulse of a plurality of radio-frequency excitation pulses in the MRI scan, sub-sampling a plurality of ky-kz planes using a three-dimensional (3D) echo planar imaging (EPI) sequence, by:
sub-sampling a plurality of phase encodings using a coherent sub-sampling in a ky dimension, wherein the coherent sub-sampling corresponds to a number of the plurality of radio-frequency excitation pulses; and
sub-sampling, for each of the plurality of phase encodings in the ky dimension, one or more phase encodings in a kz dimension, wherein the plurality of ky-kz planes represent a sub-sampling pattern in the ky-kz space, using an incoherent sub-sampling in the kz dimension.

2. The method of claim 1, wherein the incoherent sub-sampling in the kz dimension corresponds to a random sub-sampling pattern.

3. The method of claim 1, wherein the plurality of phase encodings in the kz dimension are a same plurality of phase encoding values in the kz dimension for each of the plurality of phase encodings in the ky dimension for a single radio-frequency excitation pulse.

4. The method of claim 1, wherein the plurality of phase encodings in the kz dimension are a same plurality of phase encoding values in the kz dimension for each of the plurality of phase encodings in the ky dimension for all of the plurality of radio-frequency excitation pulses.

5. The method of claim 1, wherein the incoherent sub-sampling is based on a one-dimensional variable density Poisson disk in the kz dimension.

6. The method of claim 3, wherein the incoherent sub-sampling is based on a one-dimensional variable density Poisson disk in the kz dimension.

7. The method of claim 4, wherein the incoherent sub-sampling is based on a one-dimensional variable density Poisson disk in the kz dimension.

8. The method of claim 1, wherein the plurality of ky-kz planes represent a sub-sampling pattern having a density in the ky dimension that varies as a function of kz.

9. The method of claim 8, wherein the incoherent sub-sampling is based on a two-dimensional variable density Poisson disk in the ky dimension and the kz dimension in at least a portion of a ky-kz space.

10. The method of claim 3, wherein the plurality of ky-kz planes represent a sub-sampling pattern having a density in the ky dimension that varies as a function of kz.

11. The method of claim 4, wherein the plurality of ky-kz planes represent a sub-sampling pattern having a density in the ky dimension that varies as a function of kz.

12. A magnetic resonance imaging (MRI) system comprising:
a magnet system configured to generate a polarizing magnetic field about at least a portion of a subject arranged in the MRI system;
a plurality of gradient coils configured to apply gradient pulses to the polarizing magnetic field;
a radio frequency (RF) system configured to apply an RF field to the subject and to acquire magnetic resonance (MR) image data therefrom;
a processing device; and
a memory storage device comprising instructions executable by the processing device to:
for each radio-frequency excitation pulse of a plurality of radio-frequency excitation pulses in the MRI scan, sub-sampling a plurality of ky-kz planes using a three-dimensional (3D) echo planar imaging (EPI) sequence by:
sub-sampling a plurality of phase encodings using a coherent sub-sampling in a ky dimension, wherein the coherent sub-sampling corresponds to a number of the plurality of radio-frequency excitation pulses; and
sub-sampling, for each of the plurality of phase encodings in the ky dimension, one or more phase encodings in a kz dimension, wherein the plurality of ky-kz planes represent a sub-sampling pattern in the ky-kz space, using an incoherent sub-sampling in the kz dimension.

13. The MRI system of claim 12, wherein the incoherent sub-sampling in the kz dimension corresponds to a random sub-sampling pattern.

14. The MRI system of claim 12, wherein the plurality of phase encodings in the kz dimension are a same plurality of phase encoding values in the kz dimension for each of the plurality of phase encodings in the ky dimension for a single radio-frequency excitation pulse.

15. The MRI system of claim 12, wherein the plurality of phase encodings in the kz dimension are a same plurality of phase encoding values in the kz dimension for each of the plurality of phase encodings in the ky dimension for all of the plurality of radio-frequency excitation pulses.

16. The MRI system of claim 12, wherein the incoherent sub-sampling is based on a one-dimensional variable density Poisson disk in the kz dimension.

17. The MRI system of claim 14, wherein the incoherent sub-sampling is based on a one-dimensional variable density Poisson disk in the kz dimension.

18. The MRI system of claim 15, wherein the incoherent sub-sampling is based on a one-dimensional variable density Poisson disk in the kz dimension.

19. The MRI system of claim 12, wherein the plurality of ky-kz planes represent a sub-sampling pattern having a density in the ky dimension that varies as a function of kz.

20. The MRI system of claim 19, wherein the incoherent sub-sampling is based on a two-dimensional variable density Poisson disk in the ky dimension and the kz dimension in at least a portion of a ky-kz space.
